(19) United States
(12) Patent Application Publication
(10) Pub. No.: US 2012/0253431 A1
(43) Pub. Date: Oct. 4, 2012

(54) HEMISPATIAL NEGLECT TREATMENT CLIPBOARD

(76) Inventors: Jennifer Papa, Boston, MA (US); Frank Papa, Newington, CT (US); Diane Papa, Newington, CT (US)

(21) Appl. No.: 13/074,084

(22) Filed: Mar. 29, 2011

(51) Int. Cl. A61N 5/06

(52) U.S. Cl. 607/90

(57) ABSTRACT

A hemispatial neglect clipboard, the clipboard comprising: a board, the board comprising a front-side, and a back-side, a right edge and a left edge; a clip attached to generally the top of the board; at least one light located near either the right edge or left edge of the board; an on/off switch located on the front-side of the board; a power supply in signal communication with the on/off switch and the at least one light.

### TECHNICAL FIELD

The present invention relates to a clipboard for the treatment of patients with hemispatial neglect, and more specifically relates to a clipboard which provides for visual stimulation on either a left or a right side of the clipboard.

### BACKGROUND

Hemispatial neglect, also called hemiagnosia, hemineglect, unilateral neglect, spatial neglect or neglect syndrome is a neuropsychological condition in which, after damage to one hemisphere of the brain, a deficit in attention to and awareness of one side of space is observed. Hemispatial neglect is very commonly contralateral to the damaged hemisphere, but instances of ipsilesional neglect (on the same side as the lesion) have been reported.

Hemispatial neglect results most commonly from brain injury to the right cerebral hemisphere, causing visual neglect of the left-hand side of space. Right-sided spatial neglect is rare because there is redundant processing of the right space by both the left and right cerebral hemispheres, whereas in most left-dominant brains the left space is only processed by the right cerebral hemisphere. Although most strikingly affecting visual perception (visual neglect'), neglect in other forms of perception can also be found, either alone, or in combination with visual neglect.

For example, a stroke affecting the right parietal lobe of the brain can lead to neglect for the left side of the visual field, causing a patient with neglect to behave as if the left side of sensory space is nonexistent (although they can still turn left). In an extreme case, a patient with neglect might fail to eat the food on the left half of their plate, even though they complain of being hungry. If someone with neglect is asked to draw a clock, their drawing might show only the numbers 12 and 1 to 6, the other side being distorted or left blank. Neglect patients may also ignore the contralesional side of their body, shaving or adding make-up only to the non-neglected side.

Brain areas in the parietal and frontal lobes are associated with the deployment of attention (internally, or through eye movements, head turns or limb reaches) into contralateral space. Neglect is most closely related to damage to the temporo-parietal junction and posterior parietal cortex. The lack of attention to the left side of space can manifest in the visual, auditory, proprioceptive, and olfactory domains. Although hemispatial neglect often manifests as a sensory deficit (and is frequently co-morbid with sensory deficit), it is essentially a failure to pay sufficient attention to sensory input. This is particularly evident in a related, but separate, condition known as extinction. A patient with extinction following right-hemisphere damage will successfully report the presence of an object in left space when it is the only object present. However, if the same object is presented simultaneously with an object in right space, the patient will only report the object on the right.

Neglect patients can also have problems with “interior” left information, since cases have been reported in which the patient when asked to describe something he knew before his brain damage describes only its right part.

Although hemispatial neglect has been identified following left hemisphere damage (resulting in the neglect of the right side of space), it is most common after damage to the right hemisphere. This disparity is thought to reflect the fact that the right hemisphere of the brain is specialized for spatial perception and memory, whereas the left hemisphere is specialized for language—there is redundant processing of the right visual fields by both hemispheres. Hence the right hemisphere is able to compensate for the loss of left hemisphere function, but not vice versa.

There are few treatment apparatuses that are inexpensive and easy to use to help treat patients with hemispatial neglect.

Thus, there is a need for an apparatus can assist in treating people with hemispatial neglect.

### SUMMARY OF THE INVENTION

The disclosed invention relates to a hemispatial neglect clipboard, the clipboard comprising: a board, the board comprising a front-side, and a back-side, a right edge and a left edge; a clip attached to generally the top of the board; at least one light located near either the right edge or left edge of the board; an on/off switch located on the front-side of the board; a power supply in signal communication with the on/off switch and the at least one light.

### BRIEF DESCRIPTION OF THE DRAWINGS

The present disclosure will be better understood by those skilled in the pertinent art by referencing the accompanying drawings, where like elements are numbered alike in the figures, in which:

FIG. 1 is a front view of the disclosed clipboard;

FIG. 2 is a rear schematic view of the disclosed clipboard;

FIG. 3 is a front view of another embodiment of the disclosed clipboard.

### DETAILED DESCRIPTION

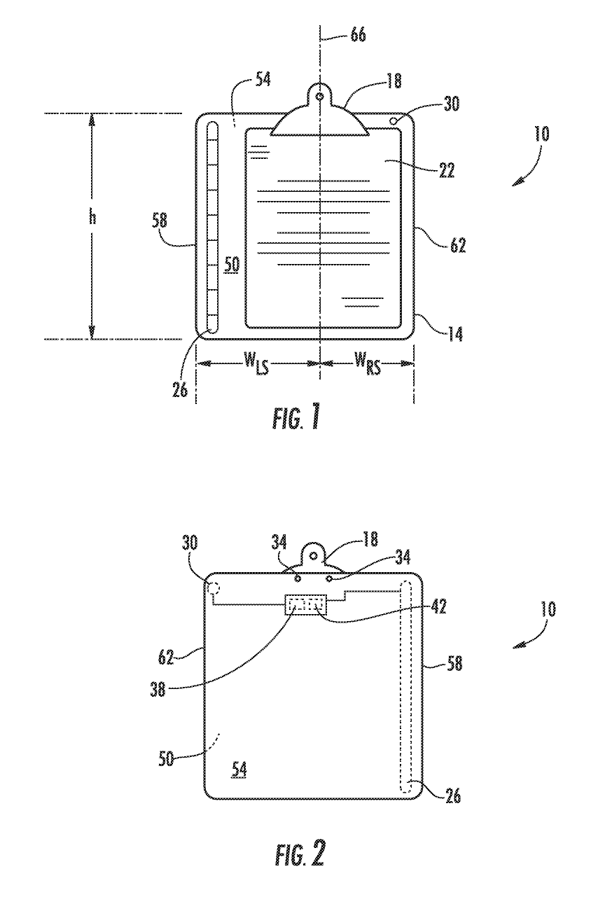

FIG. 1 front view of one embodiment of the disclosed clipboard 10. A board 14 is shown with a clip 18 attached near the top of the board. A sheet of paper 22 is being held in the clipboard 10, but one of ordinary skill in the art will recognize that many types of reading material may be held in the clipboard, including but not limited to sheets of paper, notebooks, folders, magazines, books, pamphlets, maps, photographs, drawings, pictures, playing cards, index cards, etc. The board 14 comprises a front-side 50, a back-side 54, a left edge 58, and a right edge 62. A column 26 of lights are located along the left side of the board, generally adjacent or near to the left edge 58. It should be noted, that unlike prior art clipboards, the disclosed board 14 may have extra space on the side of the board 14 with the column 26 of lights. Thus, if a vertical centerline 66 goes through the center of the clip, the left edge 58 and right edge 62 would generally be equidistance from the centerline 66 in a prior art standard clipboard. However, in the disclosed clipboard 10, the left edge is a distance W_{LS} from the centerline 66, and the right edge is a distance W_{RS} from the centerline. In one embodiment of the disclosed board 14, W_{LS} will be greater than W_{RS}. W_{LS}−W_{RS} may range from about 0 inches to about 5″. Of course, for a right neglect clipboard, there may be extra width on the right side of the board 14, and in that case, W_{RS} would be greater than W_{LS}. One of ordinary skill in the art will recognize that having the column 26 of lights located on the left side of the board 14 will primarily assist in the treatment of patients with left side neglect. The disclosed invention will include clipboards having a column of light located on the right side of the board, for those cases where the patient may have right side neglect. In one embodiment, the light source that comprise the column 26 of lights may be any suitable light source, including but not limited to: light emitting diode (LED), organic light emitting diode (OLED), Polymer light-emitting diode, Solid-state lighting, LED lamp, Electroluminescent sheet, Electroluminescent wire, incandescent lighting, fluorescent lighting; Compact fluorescent lighting, Inductive lighting, Hollow cathode lamp, Neon and argon lamps, Plasma lamps, and Xenon flash lamps. The column 26 of lights may comprise a single light source, or, as shown, a plurality of light sources, such as ten (10) LEDs. Of course, less than or more than ten (10) sources of light may be used to make up the column 26 of lights. In other embodiments, two or more columns of light may be located near one another on one side of the board. An on/off switch 30 is also located on the board 14. In this embodiment, the on/off switch is located on the upper right hand corner of the board 14; the switch 30 may be located at other positions on the board, or on the back-side of the board 14 or even on the clip 18.

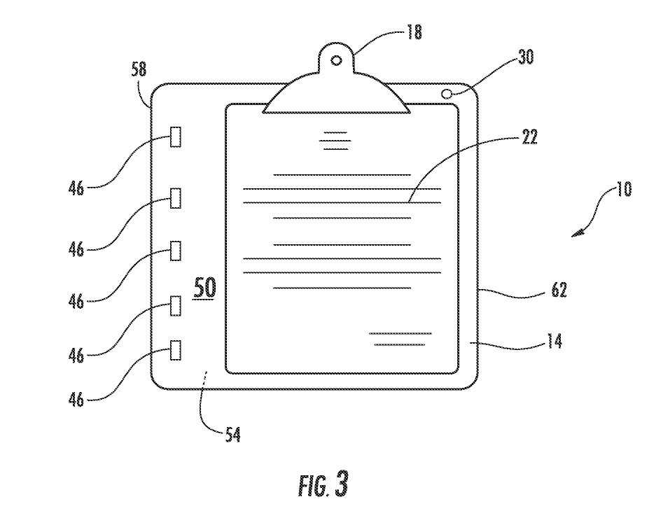

FIG. 2 is a schematic view of the back-side of the clipboard 10. The column of lights 26 is shown in dotted line because in this view it is on the other side of the board 14. Two connecting means 34 are shown attaching the clip 18 to the board 14. The on/off switch 30 is in signal communication with a power supply 38. The power supply 38 is in signal communication with the column of lights 26. In another embodiment, an optional CPU 42 may be in signal communication with the power supply 38, on/off switch 30, and the column of lights 26.

FIG. 3 is another embodiment of the disclosed clipboard 10. In this embodiment, rather than a single column 26 of lights, there may be one or more discrete lights 46.

During use, a patient may be given the clipboard 10 with reading material clipped to the board. Once the on/off switch 30 is turned on, the column of lights 26 (or the one or more discrete lights 46) will turn on. The disclosed clipboard 10 will provide visual stimulation to the left side of the reading material via the column of lights 26 (or the one or more discrete lights 46) on the left side (in this embodiment) of the clipboard. This visual cueing encourages the patient to learn to scan to the left side of the reading material during writing and reading activities, reinforcing proper scanning technique. In one embodiment, the column of lights 26 may continuously blink on and off. In one embodiment, the column of lights 26 may be of one color, such as but not limited to red, white, green blue, orange, yellow, brown, purple. In another embodiment, the light may remain continuously on while the light is turned on. In another embodiment, each of the plurality of light sources that comprise the column of lights 26 may each be a different color, or may all be the same color. In still another embodiment, the cpu may be programmed to blink the lights at different rates, allow the column of lights blink in various patterns, such as, but not limited to: giving the appearance of a light traveling up or down the column, or having two lights start at opposite ends of the column and move towards the middle, and bounce off each other and back to the opposite ends of the column, and repeat. Of course, one of ordinary skill will recognize that these different embodiments can also apply to the embodiment with the one or more discrete lights 46.

The disclosed invention has many advantages. It is a simple and inexpensively manufactured device. The blinking lights on one side (either left or right) of the reading material can assist in the treatment of patients with hemispatial neglect (either left neglect or right neglect). The color of lights, the rate of blinking, the patterns of blinking lights can all be changed to accommodate the latest in treatment methods for those suffering from hemispatial neglect.

It should be noted that the terms “first”, “second”, and “third”, and the like may be used herein to modify elements performing similar and/or analogous functions. These modifiers do not imply a spatial, sequential, or hierarchical order to the modified elements unless specifically stated.

While the disclosure has been described with reference to several embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiments disclosed as the best mode contemplated for carrying out this disclosure, but that the disclosure will include all embodiments falling within the scope of the appended claims.

What is claimed is:

1. A hemispatial neglect clipboard, the clipboard comprising:
a board, the board comprising a front-side, and a back-side, a right edge and a left edge;
a clip attached to generally the top of the board;
at least one light located near either the right edge or left edge of the board;
an on/off switch located on the front-side of the board;
a power supply in signal communication with the on/off switch and the at least one light.

2. The hemispatial neglect clipboard of claim 1, further comprising a CPU in signal communication with the power supply, the on/off switch, and the at least one light.

3. The hemispatial neglect clipboard of claim 1, wherein the at least one light located near either the right edge or left edge of the board, comprises a column of a plurality of lights located near the left edge of the board.

4. The hemispatial neglect clipboard of claim 1, wherein the at least one light located near either the right edge or left edge of the board, comprises a plurality of discrete lights located near the left edge of the board.

5. The hemispatial neglect clipboard of claim 1, wherein the at least one light is an LED.

6. The hemispatial neglect clipboard of claim 1, wherein the at least one light is a red led light.

7. The hemispatial neglect clipboard of claim 1, wherein the at least one light is selected from the group consisting of a light emitting diode (LED), an organic light emitting diode (OLED), a polymer light-emitting diode, a solid-state lighting, an LED lamp, an electroluminescent sheet, an electroluminescent wire, an incandescent light, a fluorescent light; a compact fluorescent light, an inductive light, a hollow cathode lamp, a neon lamp, an argon lamp, a plasma lamp, and a xenon flash lamp.

8. The hemispatial neglect clipboard of claim 1, wherein the distance from the right edge of the board to a vertical centerline through the center of the clip is W_{RS}, and the distance from the left edge of the board to the vertical centerline through the center of the clip is W_{LS}, and W_{LS} is greater than W_{RS}.

9. The hemispatial neglect clipboard of claim 8, where W_{LS}−W_{RS} ranges from about 1 inch to about 5 inches.

10. The hemispatial neglect clipboard of claim 3, where the plurality of lights are configured to flash in a pattern.

11. The hemispatial neglect clipboard of claim 4, where the plurality of discrete lights are configured to flash in a pattern.
